(19) United States
(12) Patent Application Publication
(10) Pub. No.: US 2025/0235336 A1
(43) Pub. Date: Jul. 24, 2025

(54) DEVICE AND METHOD FOR COMPRESSING AND LOADING A STENT

(71) Applicant: BOSTON SCIENTIFIC SCIMED, INC., Maple Grove, MN (US)

(72) Inventors: Levi Joel Wolterstorff, Saint Paul, MN (US); Eric Michael Petersen, Maple Grove, MN (US); Joseph Edward Adriaens, Elk River, MN (US)

(73) Assignee: BOSTON SCIENTIFIC SCIMED, INC., MAPLE GROVE, MN (US)

(21) Appl. No.: 19/035,320

(22) Filed: Jan. 23, 2025

Related U.S. Application Data
(60) Provisional application No. 63/624,381, filed on Jan. 24, 2024.

(51) Int. Cl. A61F 2/95

(52) U.S. Cl. CPC A61F 2/9524

(57) ABSTRACT

A device for radially compressing a stent includes a housing and a first iris including a first circumferential ring and a first plurality of arms extending inward from the ring, each arm defining a first outer hinge point and a first inner hinge point. Rotation of the ring in a first direction is configured to shift the inner hinge point from a first side to a second side of a first radius extending through a central longitudinal axis of the iris and the first outer hinge point. A radially outward force exerted against the arms biases the ring in a first direction in a first configuration and in a second direction in a second configuration. A method of loading a stent includes inserting a stent into the iris, and rotating the ring to shift the arms from the first to the second configuration.

### CROSS-REFERENCE TO RELATED APPLICATIONS

This application claims the benefit of priority of U.S. Provisional Application No. 63/624,381 filed Jan. 24, 2024, the entire disclosure of which is hereby incorporated by reference.

### TECHNICAL FIELD

The present disclosure pertains to medical devices, systems, and methods for manufacturing and/or using medical devices and/or systems. More particularly, the present disclosure pertains to a device and/or method for radially compressing and/or loading a stent and/or a stent device such as a replacement heart valve implant.

### BACKGROUND

Conventional stent crimping devices have been used throughout the medical device industry to crimp balloon expandable stents, self-expanding stents, replacement heart valve implants, etc. Conventional stent crimping devices have complex arrangements of multiple parts and/or difficult assemblies and rely upon complex interactions between many moving parts. The cost of a conventional stent crimping device may be significant due to the aforementioned complexity and number of components required and/or used in a conventional stent crimping device. Of the known devices and methods for compressing and/or crimping stents, each has certain advantages and disadvantages. There is an ongoing need for alternative devices and/or methods for compressing stents, stent devices, and/or other medical implants that may include a stent, such as but not limited to replacement heart valve implants.

### SUMMARY

In one example, a device for radially compressing a stent may comprise a housing including a central opening, and a first iris positioned adjacent the housing. The first iris may include a first circumferential ring positioned coaxially relative to the central opening and a first plurality of arms extending radially inward from the first circumferential ring. Each arm of the first plurality of arms may define a first outer hinge point and a first inner hinge point disposed radially inward relative to the first outer hinge point. Rotation of the first circumferential ring in a first circumferential direction is configured to shift the first inner hinge point of a first arm of the first plurality of arms from a first side of a first radius extending through a central longitudinal axis of the first iris and the first outer hinge point of the first arm of the first plurality of arms to a second side of the first radius.

In addition or alternatively to any example disclosed herein, a radially outward force exerted against the first arm of the first plurality of arms urges the first circumferential ring in a second circumferential direction opposite the first circumferential direction when the first inner hinge point of the first arm of the first plurality of arms is disposed on the first side of the first radius.

In addition or alternatively to any example disclosed herein, a radially outward force exerted against the first arm of the first plurality of arms urges the first circumferential ring in the first circumferential direction when the first inner hinge point of the first arm of the first plurality of arms is disposed on the second side of the first radius.

In addition or alternatively to any example disclosed herein, the first inner hinge point of the first arm of the first plurality of arms is configured to move in an opposite circumferential direction from the first circumferential ring.

In addition or alternatively to any example disclosed herein, a first axis extending through the first inner hinge point of the first arm of the first plurality of arms and the first outer hinge point of the first arm of the first plurality of arms defines a first angle from the first radius to the first axis.

In addition or alternatively to any example disclosed herein, when the first inner hinge point of the first arm of the first plurality of arms is disposed on the first side of the first radius the first angle is negative, and when the first inner hinge point of the first arm of the first plurality of arms is disposed on the second side of the first radius the first angle is positive.

In addition or alternatively to any example disclosed herein, the first plurality of arms defines a first central opening positioned coaxially relative to the central opening of the housing.

In addition or alternatively to any example disclosed herein, rotation of the first circumferential ring relative to the housing changes a size of the first central opening.

In addition or alternatively to any example disclosed herein, the first outer hinge point of each arm of the first plurality of arms is defined by a first living hinge coupling the arm to the first circumferential ring.

In addition or alternatively to any example disclosed herein, the first inner hinge point is defined by a second living hinge disposed at an opposite end of an elongated link portion extending from the first living hinge to the second living hinge.

In addition or alternatively to any example disclosed herein, the first plurality of arms is monolithically formed with the first circumferential ring from a single piece of material.

In addition or alternatively to any example disclosed herein, a device for radially compressing a stent may comprise a housing including a central opening, and a first iris positioned adjacent the housing. The first iris may include a first circumferential ring positioned coaxially relative to the central opening of the housing and a first plurality of arms extending radially inward from the first circumferential ring. The first plurality of arms may define a first central opening positioned coaxially relative to the central opening of the housing. The first plurality of arms may be configured to shift between a first configuration of the first plurality of arms and a second configuration of the first plurality of arms via rotation of the first circumferential ring relative to the housing. A radially outward force exerted against the first plurality of arms may bias the first circumferential ring in a first direction in the first configuration of the first plurality of arms and may bias the first circumferential ring in a second direction opposite the first direction in the second configuration of the first plurality of arms.

In addition or alternatively to any example disclosed herein, in the first configuration of the first plurality of arms, the first plurality of arms defines a first size of the first central opening, and in the second configuration of the first plurality of arms, the first plurality of arms defines a second size of the first central opening less than the first size of the first central opening.

In addition or alternatively to any example disclosed herein, each arm of the first plurality of arms engages at least one other arm of the first plurality of arms as the first plurality of arms shifts from the first configuration of the first plurality of arms to the second configuration of the first plurality of arms.

In addition or alternatively to any example disclosed herein, the device may further comprise a second iris axially offset from the first iris.

In addition or alternatively to any example disclosed herein, the second iris includes a second circumferential ring positioned coaxially relative to the central opening of the housing and a second plurality of arms extending radially inward from the second circumferential ring.

In addition or alternatively to any example disclosed herein, the second plurality of arms defines a second central opening positioned coaxially relative to the central opening of the housing.

In addition or alternatively to any example disclosed herein, the second plurality of arms is configured to shift between a first configuration of the second plurality of arms and a second configuration of the second plurality of arms via rotation of the second circumferential ring relative to the housing.

In addition or alternatively to any example disclosed herein, a radially outward force exerted against the second plurality of arms biases the second circumferential ring in the first direction in the first configuration of the second plurality of arms and biases the second circumferential ring in the second direction in the second configuration of the second plurality of arms.

In addition or alternatively to any example disclosed herein, in the first configuration of the second plurality of arms, the second plurality of arms defines a first size of the second central opening, and in the second configuration of the second plurality of arms, the second plurality of arms defines a second size of the second central opening less than the first size of the second central opening.

In addition or alternatively to any example disclosed herein, a method of loading a stent into a sheath may comprise inserting a stent in a first configuration into a first iris such that a first end portion of the stent is disposed outside of the first iris, wherein the first iris includes a first circumferential ring and a first plurality of arms extending radially inward from the first circumferential ring to define a first central opening; and rotating the first circumferential ring relative to a housing disposed about the first circumferential ring to shift the first plurality of arms from a first configuration to a second configuration, wherein the first central opening has a first size in the first configuration and a second size in the second configuration less than the first size. In the second configuration of the first plurality of arms, a first portion of the stent disposed within the first iris is radially compressed and exerts a radially outward force against the first plurality of arms, wherein the first portion of the stent is disposed immediately adjacent the first end portion of the stent. In the second configuration of the first plurality of arms, the radially outward force against the first plurality of arms biases the first plurality of arms toward the second configuration of the first plurality of arms.

In addition or alternatively to any example disclosed herein, the method may further comprise positioning a sheath proximate the first iris with the first plurality of arms in the second configuration and the first portion of the stent radially compressed within the first iris such that the first end portion of the stent is disposed within the sheath; rotating the first circumferential ring relative to the housing to shift the first plurality of arms from the second configuration of the first plurality of arms to the first configuration of the first plurality of arms; and moving the sheath into the first iris over the stent such that the first portion of the stent that was disposed within the first iris is disposed within the sheath.

In addition or alternatively to any example disclosed herein, the sheath has an inner diameter less than an outer diameter of the first portion of the stent when the stent is in the first configuration.

In addition or alternatively to any example disclosed herein, inserting the stent further includes inserting the stent in the first configuration into the first iris and a second iris axially offset from the first iris, wherein the second iris includes a second circumferential ring and a second plurality of arms extending radially inward from the second circumferential ring to define a second central opening.

In addition or alternatively to any example disclosed herein, the method may further comprise rotating the second circumferential ring relative to the housing to shift the second plurality of arms from a first configuration of the second plurality of arms to a second configuration of the second plurality of arms, wherein the second central opening has a first size in the first configuration and a second size in the second configuration less than the first size. In the second configuration of the second plurality of arms, a second portion of the stent disposed within the second iris is radially compressed and exerts a radially outward force against the second plurality of arms. In the second configuration of the second plurality of arms, the radially outward force against the second plurality of arms biases the second plurality of arms toward the second configuration of the second plurality of arms.

In addition or alternatively to any example disclosed herein, the method may further comprise positioning a sheath proximate the first iris with the first plurality of arms in the second configuration of the first plurality of arms and the first portion of the stent radially compressed within the first iris and the second portion of the stent radially compressed within the second iris such that the first end portion of the stent is disposed within the sheath; rotating the first circumferential ring relative to the housing to shift the first plurality of arms from the second configuration of the first plurality of arms to the first configuration of the first plurality of arms; moving the sheath into the first iris over the stent such that the first portion of the stent that was disposed within the first iris is disposed within the sheath; rotating the second circumferential ring relative to the housing to shift the second plurality of arms from the second configuration of the second plurality of arms to the first configuration of the second plurality of arms; and moving the sheath into the second iris over the stent such that the second portion of the stent that was disposed within the second iris is disposed within the sheath.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIGS. 1-2 illustrates selected aspects of a device for radially compressing a stent;

FIG. 3 is an exploded view illustrating selected aspects of the device of FIGS. 1-2;

FIG. 4 illustrates selected aspects related to the function of the device of FIGS. 1-3;

FIG. 4A is a detailed view illustrating selected aspects of FIG. 4;

FIGS. 5-6 illustrate selected aspects related to the function of the device of FIGS. 1-3;

FIG. 6A is a detailed view illustrating selected aspects of FIG. 6;

FIGS. 7-8 illustrate selected aspects related to the function of the device of FIGS. 1-3;

FIG. 8A is a detailed view illustrating selected aspects of FIG. 8;

FIGS. 9-13 are schematic partial cross-sectional views illustrating selected aspects of a method of radially compressing a stent using the device of FIGS. 1-3.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate example embodiments of the disclosure but not limit the disclosure. Those skilled in the art will recognize that the various elements described and/or shown may be arranged in various combinations and configurations without departing from the scope of the disclosure. However, in the interest of clarity and ease of understanding, every feature and/or element may not be shown in each drawing.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered the greatest possible dimension measured according to the intended usage, while a “minimum extent” may be considered the smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the arm”, “the aperture”, or other features may be equally referred to all instances and quantities beyond one of said feature unless clearly stated to the contrary. As such, it will be understood that the following discussion may apply equally to any and/or all of the components for which there are more than one within the device, etc. unless explicitly stated to the contrary.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below. For the purpose of this disclosure, the discussion below is directed toward a device and method for radially compressing a stent and will be so described in the interest of brevity. This, however, is not intended to be limiting as the skilled person will recognize that the following discussion may also apply to stent devices or medical implants including a stent with no or minimal changes to the structure and/or scope of the disclosure. Similarly, the devices and methods disclosed herein may have applications and uses for other medical devices.

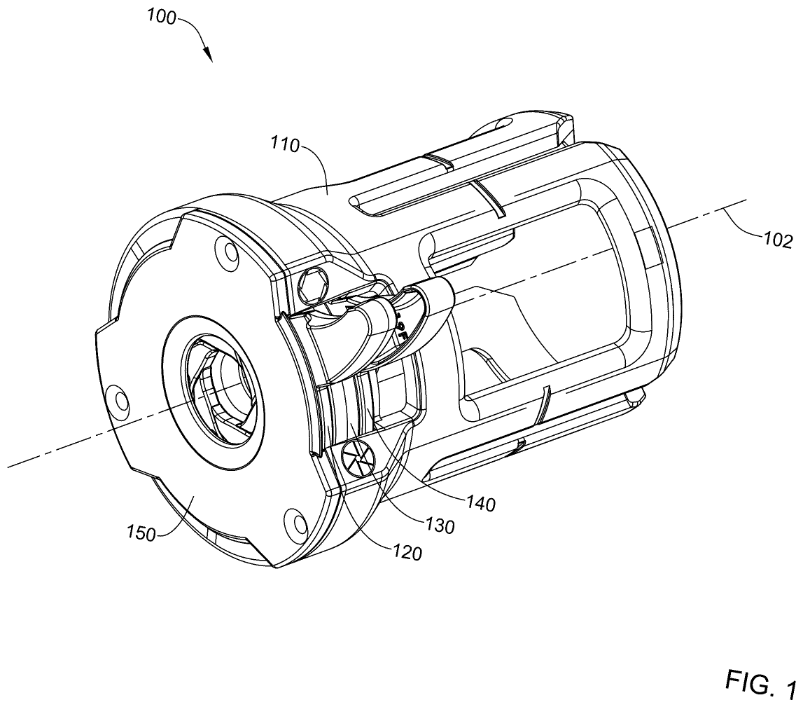

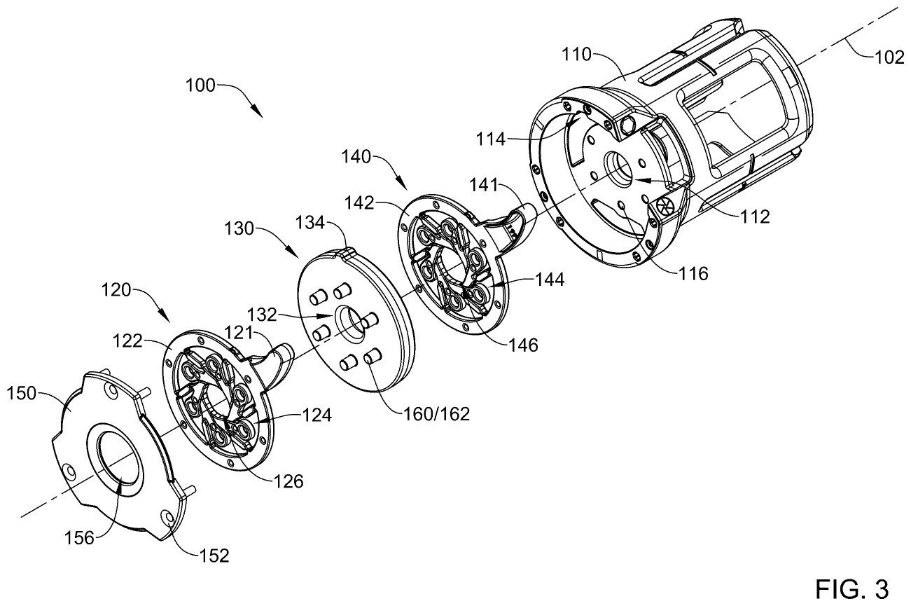

FIGS. 1-3 illustrate aspects of a device 100 for radially compressing a stent. It should be noted that some elements of the device 100 have been omitted from illustration to improve clarity. The device 100 may comprise a housing 110 including a central opening 112 (e.g., FIG. 3) aligned with and/or disposed coaxially with a central longitudinal axis 102 of the device 100 and/or the housing 110. In some embodiments, the device 100 may comprise a first iris 120 positioned adjacent the housing 110. In at least some embodiments, the first iris 120 may be positioned at least partially within the housing 110. In some embodiments, the device 100 may comprise a spacer plate 130 positioned adjacent the housing 110. In at least some embodiments, the spacer plate 130 may be positioned at least partially within the housing 110. In some embodiments, the device 100 may comprise a second iris 140 positioned adjacent the housing 110. In at least some embodiments, the second iris 140 may be positioned at least partially within the housing 110. In some embodiments, the device 100 may comprise a cover plate 150 positioned adjacent the housing 110. In some embodiments, the cover plate 150 may be positioned at least partially within the housing 110.

In some embodiments, the spacer plate 130 may be non-rotatable relative to the housing 110. In some embodiments, the spacer plate 130 may engage the housing 110 as discussed herein to prevent relative rotation therebetween. In some embodiments, the second iris 140 may be axially offset from the first iris 120. In some embodiments, the spacer plate 130 may be disposed between the first iris 120 and the second iris 140. In some embodiments, the spacer plate 130 may be configured to axially space apart the first iris 120 from the second iris 140. Other configurations are also contemplated.

In some embodiments, the cover plate 150 may be removably secured to the housing 110. In some embodiments, the cover plate 150 may be non-rotatable relative to the housing 110. In one example, the cover plate 150 may be removably secured to the housing 110 using one or more fasteners (not shown). In another example, the cover plate 150 and/or one or more protrusions extending from the cover plate 150 may be configured to engage one or more slots or other features formed in the housing 110 to removably secure the cover plate 150 to the housing 110. Other configurations are also contemplated.

In some embodiments, the first iris 120 may be movable with respect to the housing 110, the spacer plate 130, and/or the cover plate 150. In some embodiments, at least a portion of the first iris 120 may be configured to rotate relative to the housing 110, the spacer plate 130, and/or the cover plate 150. In some embodiments, the second iris 140 may be movable with respect to the housing 110, the spacer plate 130, and/or the cover plate 150. In some embodiments, at least a portion of the second iris 140 may be configured to rotate relative to the housing 110, the spacer plate 130, and/or the cover plate 150. In some embodiments, at least a portion of the second iris 140 may be configured to rotate relative to the first iris 120. Accordingly, in at least some embodiments, the first iris 120 and the second iris 140 may be movable independently of each other. In some embodiments, the first iris 120 may be movable and/or may be configured to rotate relative to the housing 110, the spacer plate 130, and/or the cover plate 150 independently of the second iris 140. In some embodiments, the second iris 140 may be movable and/or may be configured to rotate relative to the housing 110, the spacer plate 130, and/or the cover plate 150 independently of the first iris 120.

In some embodiments, the first iris 120 and the second iris 140 may be movable and/or may be configured to rotate relative to the housing 110, the spacer plate 130, and/or the cover plate 150 together, in tandem, and/or simultaneously. In some embodiments, the first iris 120 and the second iris 140 may be movable and/or may be configured to rotate relative to the housing 110, the spacer plate 130, and/or the cover plate 150 together, in tandem, and/or simultaneously at one time, in one circumferential direction, or during a particular step of a method disclosed herein, and the first iris 120 and the second iris 140 may be movable and/or may be configured to rotate relative to the housing 110, the spacer plate 130, and/or the cover plate 150 independently of each other at another time, in another direction, or during another particular step of a method disclosed herein. Other configurations are also contemplated.

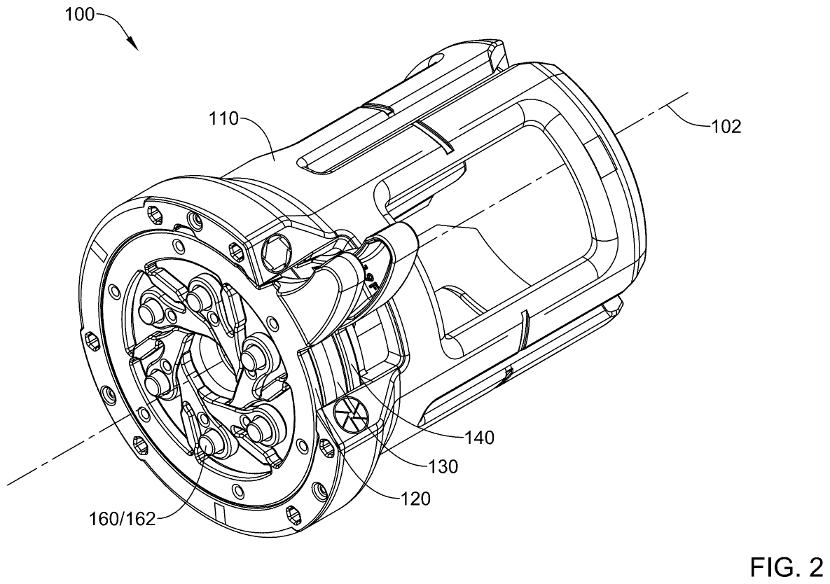

FIG. 2 illustrates selected aspects of the device 100 with the cover plate 150 removed. In some embodiments, the device 100 and/or the spacer plate 130 may include a plurality of projections 160. In some embodiments, the plurality of projections 160 may be removable from and/or configured to be disengaged from the spacer plate 130. In at least some embodiments, the plurality of projections 160 may be fixedly attached to the spacer plate 130. In some embodiments, the plurality of projections 160 may be integrally and/or monolithically formed with the spacer plate 130. In some embodiments, the plurality of projections may comprise a first plurality of projections 162 extending proximally from the spacer plate 130 and a second plurality of projections (not visible) extending distally from the spacer plate 130. The plurality of projections 160 may be configured to engage the housing 110, the first iris 120, the second iris 140, and/or the cover plate 150. In some embodiments, the first plurality of projections 162 may be configured to engage the first iris 120 and/or the cover plate 150. In some embodiments, the second plurality of projections may be configured to engage the second iris 140 and/or the housing 110. Other configurations are also contemplated.

FIG. 3 is an exploded view illustrating selected aspects of the device 100. As discussed above, the housing 110 may include the central opening 112 formed therein. In at least some embodiments, the housing 110 may include a plurality of apertures 116 configured to receive the second plurality of projections extending distally from the spacer plate 130. In some embodiments, the plurality of apertures 116 may be arranged in an array around the central opening 112.

In some embodiments, the cover plate 150 may include a cover plate opening 156. The cover plate opening 156 may be aligned with and/or may be positioned coaxially relative to the central opening 112 of the housing 110. In some embodiments, the cover plate 150 may include a plurality of openings 152 extending along a perimeter of the cover plate 150. The plurality of openings 152 may be configured to receive fasteners therein to removably secure and/or removably attach the cover plate 150 to the housing 110. The housing 110 may include a plurality of corresponding holes or recesses aligned with the plurality of openings 152 in the cover plate 150 and configured to receive the fasteners therein. In one example, the fasteners may be externally threaded screws or other threaded fasteners and the plurality of corresponding holes or recesses may include internal threads configured to threadably engage the externally threaded screws or other threaded fasteners. Other configurations and/or fastener types are also contemplated. Some suitable but non-limiting examples of materials that may be used to for the housing 110 and/or the cover plate 150, including but not limited to metals and metal alloys, composites, ceramics, polymers, and the like, are described below.

In some embodiments, the first iris 120 may include a first circumferential ring 122 positioned coaxially relative to the central opening 112 of the housing 110 and/or the central longitudinal axis 102. In some embodiments, the first circumferential ring 122 may be positioned adjacent the housing 110. In some embodiments, the first circumferential ring 122 may be positioned at least partially within the housing 110. In some embodiments, the first iris 120 and/or the first circumferential ring 122 may have a substantially circular outer perimeter.

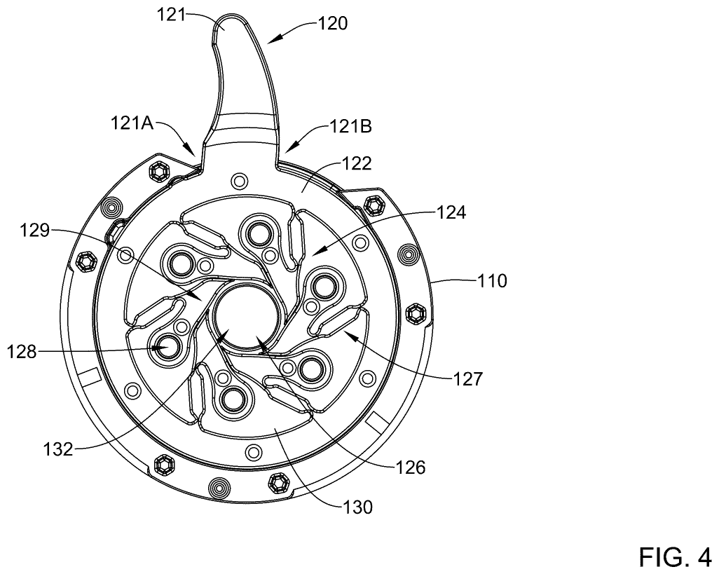

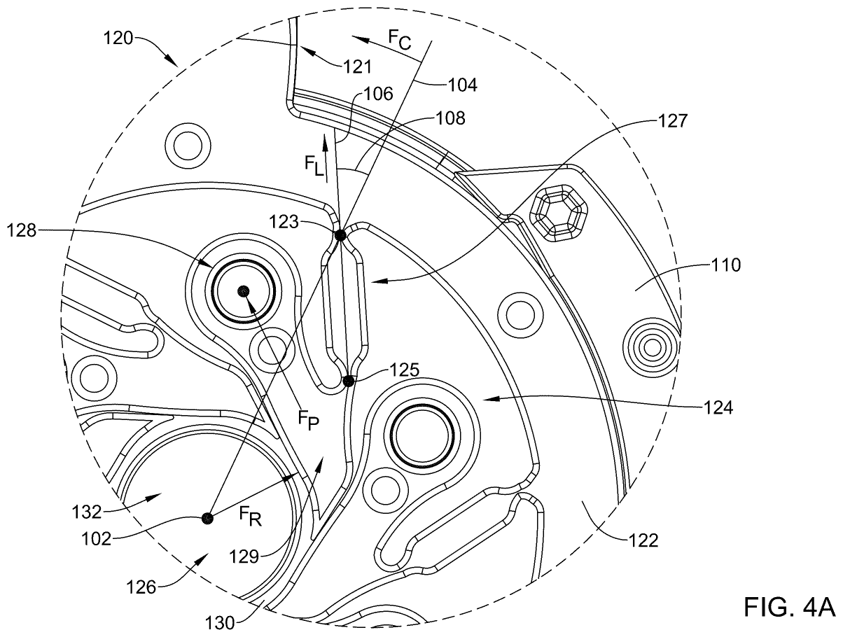

The first iris 120 may include a first plurality of arms 124 extending radially inward from the first circumferential ring 122. The first plurality of arms 124 may define a first central opening 126 of the first iris 120. The first central opening 126 may be aligned with and/or may be positioned coaxially relative to the central opening 112 of the housing 110 and/or the central longitudinal axis 102. Some aspects of the first iris 120 and/or elements thereof are shown in greater detail in FIGS. 4 and 4A.

In some embodiments, each arm of the first plurality of arms 124 may define a first outer hinge point 123 and a first inner hinge point 125 disposed radially inward relative to the first outer hinge point 123, as seen in FIG. 4A. The first outer hinge point 123 of each arm of the first plurality of arms 124 may be defined by a first living hinge coupling the arm to the first circumferential ring 122. The first inner hinge point 125 each arm of the first plurality of arms 124 each arm of the first plurality of arms 124 may be defined by a second living hinge disposed at an opposite end of an elongated link portion 127 extending from the first living hinge to the second living hinge each arm of the first plurality of arms 124.

The elongated link portion 127 of each arm of the first plurality of arms 124 may be coupled to and/or connected to the first circumferential ring 122 by the first living hinge disposed between the elongated link portion 127 and the first circumferential ring 122. In some embodiments, the elongated link portion 127 of each arm of the first plurality of arms 124 may be directly connected to the first circumferential ring 122 by the first living hinge. In some embodiments, the elongated link portion 127 of each arm of the first plurality of arms 124 may be fixedly attached to the first circumferential ring 122 by the first living hinge.

In some embodiments, a pivot end of each arm of the first plurality of arms 124 may be secured relative to the housing 110 and/or the spacer plate 130. In some embodiments, the pivot end of each arm of the first plurality of arms 124 may include an aperture 128 formed therein. In some embodiments, the plurality of projections 160 and/or the first plurality of projections 162 may extend through the aperture 128 and define a pivot point at and/or for the pivot end of each arm of the first plurality of arms 124. In some embodiments, the aperture 128 of each arm of the first plurality of arms 124 may be configured to receive and/or engage with the plurality of projections 160 and/or the first plurality of projections 162 extending from the spacer plate 130. In at least some embodiments, the pivot end of each arm of the first plurality of arms 124 may be configured to rotate about the plurality of projections 160 and/or the first plurality of projections 162 when the first circumferential ring 122 is rotated relative to the housing 110, the spacer plate 130, and/or the second iris 140.

A medial portion 129 of each arm of the first plurality of arms 124 may be configured to engage with medial portions of circumferentially adjacent arms of the first plurality of arms 124 to define the first central opening 126. In some embodiments, the medial portion 129 of each arm of the first plurality of arms 124 may be coupled to the second living hinge. In some embodiments, the second living hinge of each arm of the first plurality of arms 124 may couple the medial portion 129 of each arm of the first plurality of arms 124 to the elongated link portion 127 of each arm of the first plurality of arms 124. In some embodiments, the medial portion 129 of each arm of the first plurality of arms 124 may be directly connected to the elongated link portion 127 of each arm of the first plurality of arms 124 by the second living hinge of each arm of the first plurality of arms 124. In some embodiments, the first plurality of arms 124 may be integrally and/or monolithically formed with the first circumferential ring 122 from a single piece of material. In some embodiments, the first living hinge, the second living hinge, the elongated link portion 127, and the medial portion 129 of each arm of the first plurality of arms 124 may be integrally and/or monolithically formed with the first circumferential ring 122 from a single piece of material.

In at least some embodiments, the first living hinge and the second living hinge of each arm of the first plurality of arms 124 may be configured to resiliently flex, deflect, and/or bend to permit relative movement between portions of the first iris 120 on opposite sides of the first living hinge and/or the second living hinge. In a preferred configuration, the first iris 120 may be made from a polymeric material. Some suitable but non-limiting examples of materials that may be used to form the first iris 120, the first circumferential ring 122, the first plurality of arms 124, etc., including but not limited to metals and metal alloys, composites, ceramics, polymers, and the like, are described below.

In some embodiments, the first iris 120 may be manufactured using one or more a variety of methods. In some embodiments, the first iris 120 may be machined. In some embodiments, the first iris 120 may be cut using a waterjet. In some embodiments, the first iris 120 may be laser cut. In some embodiments, the first iris 120 may be injection molded. In some embodiments, the first iris 120 may be cast. Other methods of manufacture are also contemplated.

In some embodiments, the first iris 120 may include a first lever 121 fixedly attached to and/or extending radially outward from the first circumferential ring 122. In some embodiments, the first lever 121 may extend radially outward from the substantially circular outer perimeter of the first iris 120 and/or the first circumferential ring 122. The first lever 121 may extend radially outward along a radius extending outward from a center of the first central opening 126 and/or the central longitudinal axis 102. The first lever 121 may include a first side surface 121A facing laterally from the first radius in a first circumferential direction and a second side surface 121B disposed generally opposite the first side surface 121A, the second side surface 121B facing laterally from the first radius in a second circumferential direction generally opposite the first circumferential direction. The first side surface 121A and/or the second side surface 121B of the first lever 121 may be configured to engage and/or contact the housing 110 to stop rotation of the first iris 120 and/or the first lever 121 in the first circumferential direction and the second circumferential direction, respectively.

Returning to FIG. 3, in some embodiments, the device 100 may comprise the spacer plate 130. In some embodiments, the spacer plate 130 may include a central opening 132 formed therein. In some embodiments, the central opening 132 of the spacer plate 130 may be aligned with and/or may be positioned coaxially relative to the central opening 112 of the housing 110, the first central opening 126 of the first iris 120, and/or the central longitudinal axis 102. In some embodiments, the spacer plate 130 may be disposed axially between the first iris 120 and the housing 110 with respect to the central longitudinal axis 102. In some embodiments, the spacer plate 130 may be configured slide axially into the housing 110. The housing 110 may include a notch or a recess 114 formed in a sidewall of the housing 110. The spacer plate 130 may include a locking tab 134 projecting radially outward from an outer perimeter of the spacer plate 130. In some embodiments, the outer perimeter of the spacer plate 130 may be substantially circular. The locking tab 134 may be configured to engage with the recess 114 of the housing 110 to prevent relative rotation between the spacer plate 130 and the housing 110. In some embodiments, the spacer plate 130 may include the plurality of projections 160, the first plurality of projections 162, and/or the second plurality of projections extending axially therefrom.

In some embodiments, the device 100 may be devoid of the spacer plate 130. In some embodiments, the first iris 120 may be positioned immediately adjacent to an upward facing surface of the housing 110 defining the central opening 112. Such a configuration may be useful for radially compressing a relative short and/or limited length stent, or where only a portion of the stent needs to be radially compressed.

In some embodiments, the device 100 may comprise the second iris 140. The second iris 140 may be axially offset from the first iris 120. In some embodiments, the second iris 140 may be spaced apart from the first iris 120. In some embodiments, the second iris 140 may be spaced apart from the first iris 120 by the spacer plate 130. In some embodiments, the second iris 140 may include a second circumferential ring 142 positioned coaxially relative to the central opening 112 of the housing 110 and/or the central longitudinal axis 102. In some embodiments, the second circumferential ring 142 may be positioned adjacent the housing 110. In some embodiments, the second circumferential ring 142 may be positioned at least partially within the housing 110. In some embodiments, the second iris 140 and/or the second circumferential ring 142 may have a substantially circular outer perimeter.

The second iris 140 may include a second plurality of arms 144 extending radially inward from the second circumferential ring 142. The second plurality of arms 144 may define a second central opening 146 of the second iris 140. The second central opening 146 may be aligned with and/or may be positioned coaxially relative to the central opening 112 of the housing 110, the central opening 132 of the spacer plate 130, and/or the first central opening 126 of the first iris 120. Some aspects of the second iris 140 and/or elements thereof may be substantially the same as or similar to elements of the first iris 120 shown in FIGS. 4 and 4A, except where expressly noted otherwise, and thus are not repeated. The skilled person shall recognize that like elements and/or features of the first iris 120 and the second iris 140 may be identified using reference numerals 12x and 14x, respectively, except where expressly noted otherwise.

In some embodiments, each arm of the second plurality of arms 144 may define a second outer hinge point and a second inner hinge point disposed radially inward relative to the second outer hinge point, similar to the first outer hinge point 123 and the first inner hinge point 125 shown in FIG. 4A. The second outer hinge point may be defined by a first living hinge coupling the arm to the second circumferential ring 142. The second inner hinge point may be defined by a second living hinge disposed at an opposite end of an elongated link portion extending from the first living hinge to the second living hinge.

The elongated link portion of each arm of the second plurality of arms 144 may be coupled to and/or connected to the second circumferential ring 142 by the first living hinge disposed between the elongated link portion and the second circumferential ring 142. In some embodiments, the elongated link portion of each arm of the second plurality of arms 144 may be directly connected to the second circumferential ring 142 by the first living hinge. In some embodiments, the elongated link portion of each arm of the second plurality of arms 144 may be fixedly attached to the second circumferential ring 142 by the first living hinge.

In some embodiments, a pivot end of each arm of the second plurality of arms 144 may be secured relative to the housing 110 and/or the spacer plate 130. In some embodiments, the pivot end of each arm of the second plurality of arms 144 may include an aperture formed therein. In some embodiments, the plurality of projections 160 and/or the second plurality of projections may extend through the aperture and engage with the housing 110 and/or the plurality of apertures 116. In some embodiments, the aperture may be configured to engage with the plurality of projections 160 and/or the second plurality of projections extending from the spacer plate 130. In at least some embodiments, the pivot end of each of the second plurality of arms 144 may be configured to rotate about the plurality of projections 160 and/or the second plurality of projections when the second circumferential ring 142 is rotated relative to the housing 110, the spacer plate 130, and/or the first iris 120.

Similar to the first iris 120 shown in FIGS. 4 and 4A, a medial portion of each arm of the second plurality of arms 144 of the second iris 140 may be configured to engage with medial portions of circumferentially adjacent arms of the second plurality of arms 144 to define the second central opening 146. The medial portion of each arm of the second plurality of arms 144 may be coupled to the second living hinge. In some embodiments, the second plurality of arms 144 may be integrally and/or monolithically formed with the second circumferential ring 142 from a single piece of material. In at least some embodiments, the first living hinge and the second living hinge of each arm of the second plurality of arms 144 may be configured to resiliently flex, deflect, and/or bend to permit relative movement between portions of each arm on opposite sides of the first living hinge and/or the second living hinge. In a preferred configuration, the second iris 140 may be made from a polymeric material. Some suitable but non-limiting examples of materials that may be used to form the second iris 140, the second circumferential ring 142, the second plurality of arms 144, etc., including but not limited to metals and metal alloys, composites, ceramics, polymers, and the like, are described below.

In some embodiments, the second iris 140 may be manufactured using one or more a variety of methods. In some embodiments, the second iris 140 may be machined. In some embodiments, the second iris 140 may be cut using a waterjet. In some embodiments, the second iris 140 may be laser cut. In some embodiments, the second iris 140 may be injection molded. In some embodiments, the second iris 140 may be cast. Other methods of manufacture are also contemplated.

In some embodiments, as seen in FIG. 3 for example, the second iris 140 may include a second lever 141 fixedly attached to and/or extending radially outward from the second circumferential ring 142. In some embodiments, the second lever 141 may extend radially outward from the substantially circular outer perimeter of the second iris 140 and/or the second circumferential ring 142. The second lever 141 may extend radially outward along a second radius extending outward from a center of the second central opening 146 and/or the central longitudinal axis 102. Additionally, similar to the first iris 120 shown in FIG. 4, the second lever 141 may include a first side surface facing laterally from the second radius in the first circumferential direction and a second side surface disposed generally opposite the first side surface, the second side surface facing laterally from the second radius in the second circumferential direction generally opposite the first circumferential direction. The first side surface and/or the second side surface of the second lever 141 may be configured to engage and/or contact the housing 110 to stop rotation of the second iris 140 and/or the second lever 141 in the first circumferential direction and the second circumferential direction, respectively.

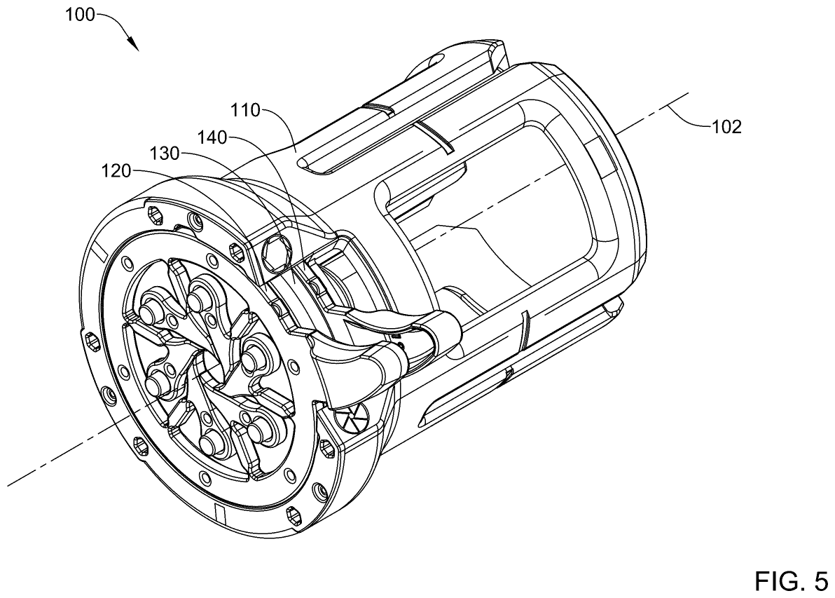

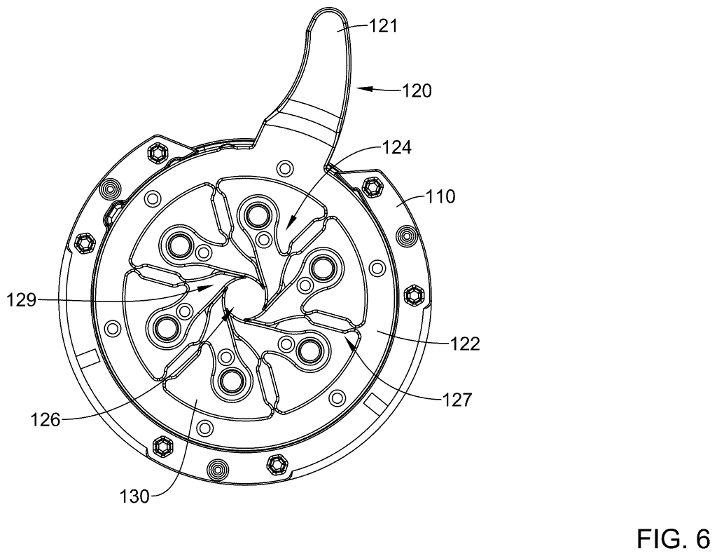

FIGS. 4-8A illustrate selected aspects related to the function and/or operation of the device 100. To improve clarity, the cover plate 150 is not shown. Each and every reference number illustrated may not be expressly discussed (and vice versa) with respect to FIGS. 5-8A, but earlier discussion related to such reference numbers (e.g., with respect to FIGS. 1-4) may apply.

FIGS. 2 and 4 illustrate the first iris 120 and/or the first plurality of arms 124 in a first configuration. The first plurality of arms 124 may be configured to shift between the first configuration of the first plurality of arms 124 and a second configuration of the first plurality of arms 124 via rotation of the first circumferential ring 122 relative to the housing 110. The first lever 121 may extend radially outward from the first circumferential ring 122 through a side opening in the housing 110. The first lever 121 may be movable and/or rotatable relative to the housing 110 within the side opening of the housing 110. In some embodiments, the first plurality of arms 124 may be self-biased toward the first configuration. The first plurality of arms 124 defines the first central opening 126. In the first configuration, the first plurality of arms 124 defines a first size of the first central opening 126.

As discussed herein, each arm of the first plurality of arms 124 may define the first outer hinge point 123 and the first inner hinge point 125. Each arm of the first plurality of arms 124 may define a pivot point at and/or within the aperture 128 (e.g., at a center of the aperture 128). Each arm and/or the medial portion 129 of each arm of the first plurality of arms 124 may be configured to pivot around its pivot point and/or its aperture 128. When a stent is disposed within the first central opening 126, the stent may apply a radially outward force F_{R} against the medial portion 129 of each arm of the first plurality of arms 124. For the purpose of discussion, when referring to the detailed views of FIGS. 4A, 6A, and 8A, discussion will refer to a first arm of the first plurality of arms 124. It shall be understood that this discussion may apply to each arm of the first plurality of arms 124.

The radially outward force F_{R} may be translated along the first arm of the first plurality of arms 124. A portion of the radially outward force F_{R} may be applied along the medial portion 129 of the first arm of the first plurality of arms 124 to the pivot point at and/or within the aperture 128 as a pivot force F_{P}. A portion of the radially outward force F_{R} may be applied along the elongated link portion 127 and/or along a first axis 106 extending through the first inner hinge point 125 and the first outer hinge point 123 as a link force F_{L}. The first iris 120 may define a first radius 104 extending through the central longitudinal axis 102 and the first outer hinge point 123. The first axis 106 may define a first angle 108 from the first radius 104 to the first axis 106. The circumferential direction of the first angle 108 from the first radius 104 may define and/or correspond to a circumferential force F_{C} transmitted to the first circumferential ring 122. The circumferential force F_{C} may urge and/or bias the first circumferential ring 122 to rotate circumferentially around the central longitudinal axis 102 independently of force applied to the first lever 121.

Similarly, while not expressly visible, the second iris 140 and/or the second plurality of arms 144 is also in a first configuration. The second plurality of arms 144 may be configured to shift between the first configuration of the second plurality of arms 144 and a second configuration of the second plurality of arms 144 via rotation of the second circumferential ring 142 relative to the housing 110. The second lever 141 may extend radially outward from the second circumferential ring 142 through the side opening in the housing 110. The second lever 141 may be movable and/or rotatable relative to the housing 110 within the side opening of the housing. In some embodiments, the second plurality of arms 144 may be self-biased toward the first configuration. The second plurality of arms 144 defines the second central opening 146. In the first configuration, the second central opening 146 has a first size. As the structures are similar, the second iris 140 may receive and/or transmit forces similarly to those of the first iris 120.

Rotation of the first circumferential ring 122 of the first iris 120 relative to the housing 110 may change a size of the first central opening 126, as seen in FIGS. 5-8. In at least some embodiments, the first plurality of arms 124 may be configured to shift between the first configuration and a second configuration via rotation of the first circumferential ring 122 relative to the housing 110. In some embodiments, rotation of the first circumferential ring 122 relative to the housing 110 may be achieved by shifting and/or rotating the first lever 121 relative to the housing 110. In some embodiments, the first plurality of arms 124 may be configured to shift between the first configuration and the second configuration via clockwise rotation of the first circumferential ring 122 and/or the first lever 121 relative to the housing 110. In the second configuration, the first plurality of arms 124 defines a second size of the first central opening 126 less than the first size. Each arm of the first plurality of arms 124 may engage at least one other arm of the first plurality of arms 124 as the first plurality of arms 124 shifts from the first configuration to the second configuration. In some embodiments, the medial portion 129 of each arm of the first plurality of arms 124 may engage with the medial portion 129 of at least one other arm of the first plurality of arms 124 as the first plurality of arms 124 shifts from the first configurations to the second configuration to define the first central opening 126. In some embodiments, the medial portion 129 of each arm of the first plurality of arms 124 may engage with the medial portions of circumferentially and/or immediately adjacent arms of the first plurality of arms 124 as the first plurality of arms 124 shifts from the first configurations to the second configuration to define the first central opening 126.

Similarly, rotation of the second circumferential ring 142 of the second iris 140 relative to the housing 110 may change a size of the second central opening 146. In at least some embodiments, the second plurality of arms 144 may be configured to shift between the first configuration and a second configuration via rotation of the second circumferential ring 142 relative to the housing 110. In some embodiments, rotation of the second circumferential ring 142 relative to the housing 110 may be achieved by shifting and/or rotating the second lever 141 relative to the housing 110. In some embodiments, the second plurality of arms 144 may be configured to shift between the first configuration and the second configuration via clockwise rotation of the second circumferential ring 142 and/or the second lever 141 relative to the housing 110. In the second configuration, the second plurality of arms 144 defines a second size of the second central opening 146 less than the first size. Each arm of the second plurality of arms 144 may engage at least one other arm of the second plurality of arms 144 as the second plurality of arms 144 shifts from the first configuration to the second configuration. In some embodiments, the medial portion of each arm of the second plurality of arms 144 may engage with the medial portion of at least one other arm of the second plurality of arms 144 as the second plurality of arms 144 shifts from the first configuration to the second configuration to define the second central opening 146. In some embodiments, the medial portion of each arm of the second plurality of arms 144 may engage with the medial portions of circumferentially and/or immediately adjacent arms of the second plurality of arms 144 as the second plurality of arms 144 shifts from the first configuration to the second configuration to define the second central opening 146.

In at least some embodiments, the first iris 120, the first lever 121, and/or the first plurality of arms 124 may be moved, shifted, and/or actuated independently of the second iris 140, the second lever 141, and/or the second plurality of arms 144. As such, at some times and/or under some circumstances, the first central opening 126 may be at the first size while the second central opening 146 is at the second size, and vice versa.

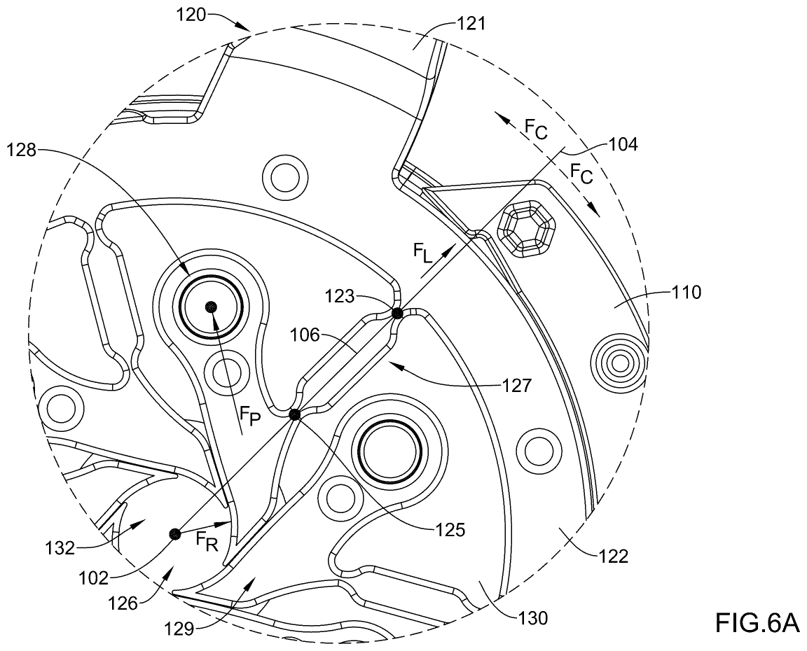

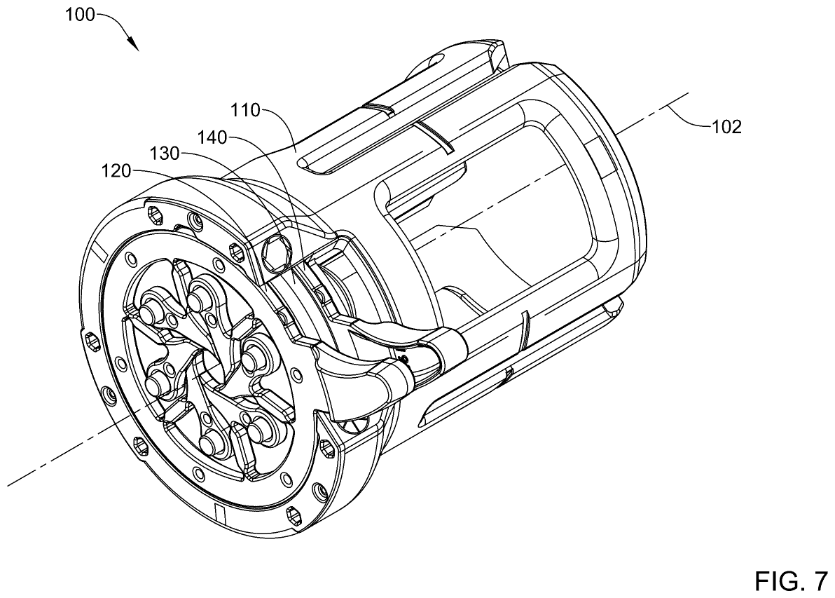

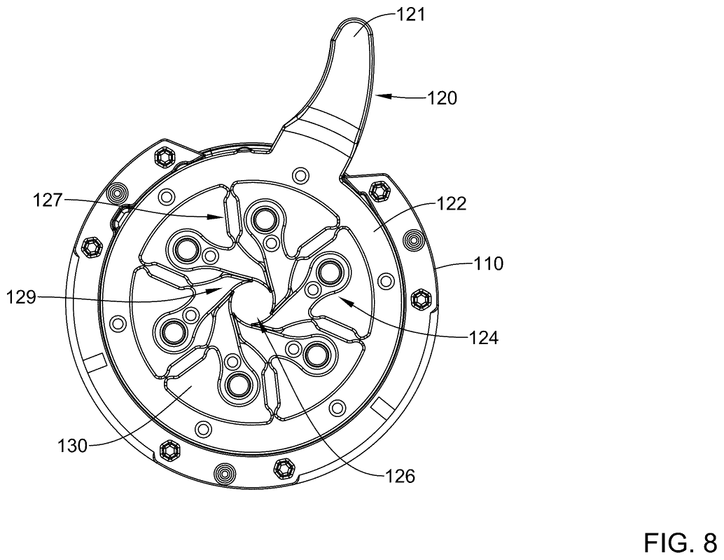

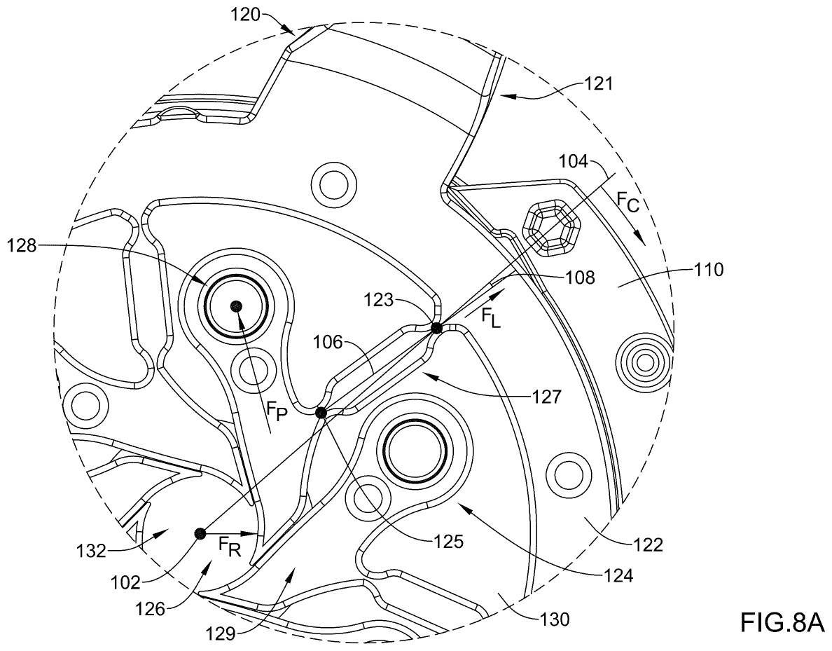

In FIGS. 5 and 6, both the first lever 121 of the first iris 120 and the second lever 141 of the second iris 140 have been rotated clockwise relative to the housing 110 to shift the first iris 120 and/or the first plurality of arms 124 and the second iris 140 and/or the second plurality of arms 144 toward and/or to the second configuration. In FIGS. 7 and 8, both the first lever 121 of the first iris 120 and the second lever 141 of the second iris 140 have been rotated clockwise relative to the housing 110 further than in FIGS. 5 and 6. In FIGS. 5 and 6, the first iris 120 and/or the first plurality of arms 124 (as well as the second iris 140 and/or the second plurality of arms 144) may be considered to be in a neutral position or a balanced position, wherein the first circumferential ring 122 (as well as the second circumferential ring 142) is unbiased in any particular circumferential direction. This occurs when the first radius 104 is aligned with and/or is coaxial with the first axis 106, as shown in FIG. 6A, and/or when the first angle 108 (e.g., FIGS. 4A, 8A) is zero. A very small circumferential force applied to the first circumferential ring 122 (or the second circumferential ring 142) in either direction may shift the first iris 120 and/or the first plurality of arms 124 (or the second iris 140 and/or the second plurality of arms 144) out of the neutral position or the balanced position, thereby reestablishing bias on the first circumferential ring 122 (or the second circumferential ring 142).

In at least some embodiments, rotation of the first circumferential ring 122 in a first circumferential direction (e.g., clockwise) may be configured to shift the first inner hinge point 125 of a first arm of the first plurality of arms 124 of the first iris 120 from a first side of the first radius 104 extending through the central longitudinal axis 102 of the first iris 120 and/or the device 100 and the first outer hinge point 123 of the first arm of the first plurality of arms 124 of the first iris 120, as shown in FIG. 4A, to a second side of the first radius 104, as shown in FIG. 8A.

As may be seen by comparing FIG. 4A to FIGS. 6A and 8A, rotation of the first lever 121, the first circumferential ring 122, and/or the first iris 120 in a first circumferential direction (e.g., clockwise) relative to the housing 110 may shift and/or move the first inner hinge point 125 in an opposite circumferential direction (e.g., a second circumferential direction opposite the first circumferential direction, counterclockwise) from the first circumferential ring 122. The reverse also applies, and rotation of the first lever 121, the first circumferential ring 122, and/or the first iris 120 in the second circumferential direction (e.g., counterclockwise) relative to the housing 110 may shift and/or move the first inner hinge point 125 in an opposite circumferential direction (e.g., the first circumferential direction opposite the second circumferential direction, clockwise) from the first circumferential ring 122.

The radially outward force F_{R} exerted against the first arm of the first plurality of arms 124 of the first iris 120 may urge and/or bias the first circumferential ring 122 in a second circumferential direction opposite the first circumferential direction (e.g., counterclockwise) when the first inner hinge point 125 of the first arm of the first plurality of arms 124 of the first iris 120 is disposed on the first side of the first radius 104. When the first inner hinge point 125 of the first arm of the first plurality of arms 124 of the first iris 120 is disposed on the first side of the first radius 104, the first angle 108 is negative (e.g., opens to the left or counterclockwise from the first radius 104). See FIG. 4A for example. As such, when the first inner hinge point 125 of the first arm of the first plurality of arms 124 of the first iris 120 is disposed on the first side of the first radius 104 and/or when the first angle 108 is negative, the radially outward force F_{R} exerted against the first arm of the first plurality of arms 124 of the first iris 120 may urge and/or bias the first iris 120 and/or the first plurality of arms 124 towards the first configuration and/or an unlocked configuration. In some embodiments, the radially outward force F_{R} exerted against the first plurality of arms 124 of the first iris 120 may urge and/or bias the first circumferential ring 122 in a first direction (e.g., the second circumferential direction, counterclockwise) in the first configuration and/or the unlocked configuration of the first plurality of arms 124.

The radially outward force F_{R} exerted against the first arm of the first plurality of arms 124 of the first iris 120 may urge and/or bias the first circumferential ring 122 in the first circumferential direction (e.g., clockwise) when the first inner hinge point 125 of the first arm of the first plurality of arms 124 of the first iris 120 is disposed on the second side of the first radius 104. When the first inner hinge point 125 of the first arm of the first plurality of arms 124 of the first iris 120 is disposed on the first side of the first radius 104, the first angle 108 is positive (e.g., opens to the right or clockwise from the first radius 104). See FIG. 8A for example. As such, when the first inner hinge point 125 of the first arm of the first plurality of arms 124 of the first iris 120 is disposed on the second side of the first radius 104 and/or when the first angle 108 is positive, the radially outward force F_{R} exerted against the first arm of the first plurality of arms 124 of the first iris 120 may urge and/or bias the first iris 120 and/or the first plurality of arms 124 towards the second configuration and/or a locked configuration. In some embodiments, the radially outward force F_{R} exerted against the first plurality of arms 124 of the first iris 120 may urge and/or bias the first circumferential ring 122 in a second direction (e.g., the first circumferential direction, clockwise) in the second configuration and/or the locked configuration of the first plurality of arms 124.

Clockwise rotation of the first lever 121 and/or the first circumferential ring 122 causes a radially outermost end and/or the first living hinge of each arm of the first plurality of arms 124 to shift in the clockwise direction as well. Since the pivot end of each arm of the first plurality of arms 124 is held in a fixed position relative to the housing 110 by engagement of the aperture 128 with the plurality of projections 160 and/or the first plurality of projections 162, the medial portion 129 of each arm of the first plurality of arms 124 will pivot about the plurality of projections 160 and/or the first plurality of projections 162 as the first lever 121 and/or the first circumferential ring 122 is rotated. The first living hinge and the second living hinge of each arm of the first plurality of arms 124 may resiliently flex, deflect, and/or bend at the first outer hinge point 123 and the first inner hinge point 125, respectively, to facilitate a pivoting movement of the first plurality of arms 124 around the pivot point and/or the aperture 128 and/or the plurality of projections 160 and/or the first plurality of projections 162. As the first lever 121 and/or the first circumferential ring 122 is rotated clockwise, each arm of the first plurality of arms 124 may be prevented from rotation commensurately and/or equally around the central longitudinal axis 102 due to the arm being fixed in position by the plurality of projections 160 and/or the first plurality of projections 162. Lengths of the various portions of each arm of the first plurality of arms 124 remain fixed and/or constant, wherein the first living hinge and the second living hinge cooperate with the elongated link portion 127 of each arm connecting the medial portion 129 to the first circumferential ring 122 to facilitate movement and/or translation of the first plurality of arms 124 from the first configuration toward and/or to the second configuration.

In some embodiments, each arm of the first plurality of arms 124 of the first iris 120 may engage at least one other arm of the first plurality of arms 124 of the first iris 120 as the first plurality of arms 124 shifts from the first configuration of the first plurality of arms 124 to the second configuration of the first plurality of arms 124. As seen when comparing FIG. 4 to FIG. 6, for example, as the first lever 121 and/or the first circumferential ring 122 is rotated clockwise and/or as the first plurality of arms 124 shifts from the first configuration to the second configuration, the medial portion 129 of each arm of the first plurality of arms 124 may shift radially inward relative to the first circumferential ring 122 and/or toward the central longitudinal axis 102, thereby changing the size of the first central opening 126 defined by the first plurality of arms 124. In the first configuration shown in FIGS. 4 and 4A, the elongated link portion 127 of each arm of the first plurality of arms 124 connected to the first circumferential ring 122 by the first living hinge and/or extending between the first living hinge and the second living hinge may be oriented at an oblique angle to the first circumferential ring 122. As the first lever 121 and/or the first circumferential ring 122 is rotated clockwise toward the second configuration, the elongated link portion 127 of each arm of the first plurality of arms 124 connected to the first circumferential ring 122 by the first living hinge and/or extending between the first living hinge and the second living hinge may shift toward an angle that is closer to normal or perpendicular to the first circumferential ring 122 (e.g., wherein the first axis 106 is aligned with the first radius 104), as seen in FIGS. 6 and 6A.

While not expressly visible in FIGS. 4 and 6, the movements and/or motions of the second plurality of arms 144 may be understood to be similar to or substantially the same as those of the first plurality of arms 124.

In some embodiments, rotation of the second circumferential ring 142 in a first circumferential direction (e.g., clockwise) may be configured to shift the first inner hinge point of a first arm of the second plurality of arms 144 of the second iris 140 from a first side of a first radius extending through the central longitudinal axis 102 of the second iris 140 and/or the device 100 and the first outer hinge point of the first arm of the second plurality of arms 144 of the second iris 140 to a second side of the first radius.

Rotation of the second lever 141, the second circumferential ring 142, and/or the second iris 140 in a first circumferential direction (e.g., clockwise) relative to the housing 110 may shift and/or move the first inner hinge point in an opposite circumferential direction (e.g., a second circumferential direction opposite the first circumferential direction, counterclockwise) from the second circumferential ring 142. The reverse also applies, and rotation of the second lever 141, the second circumferential ring 142, and/or the second iris 140 in the second circumferential direction (e.g., counterclockwise) relative to the housing 110 may shift and/or move the first inner hinge point in an opposite circumferential direction (e.g., the first circumferential direction opposite the second circumferential direction, clockwise) from the second circumferential ring 142.

A radially outward force F_{R} exerted against the first arm of the second plurality of arms 144 of the second iris 140 may urge and/or bias the second circumferential ring 142 in a second circumferential direction opposite the first circumferential direction (e.g., counterclockwise) when the first inner hinge point of the first arm of the second plurality of arms 144 of the second iris 140 is disposed on the first side of the first radius. When the first inner hinge point of the first arm of the second plurality of arms 144 of the second iris 140 is disposed on the first side of the first radius, the first angle is negative (e.g., opens to the left or counterclockwise from the first radius). As such, when the first inner hinge point of the first arm of the second plurality of arms 144 of the second iris 140 is disposed on the first side of the first radius and/or when the first angle is negative, the radially outward force F_{R} exerted against the first arm of the second plurality of arms 144 of the second iris 140 may urge and/or bias the second iris 140 and/or the second plurality of arms 144 towards the first configuration and/or an unlocked configuration. In some embodiments, the radially outward force F_{R} exerted against the second plurality of arms 144 of the second iris 140 may urge and/or bias the second circumferential ring 142 in a first direction (e.g., the second circumferential direction, counterclockwise) in the first configuration and/or the unlocked configuration of the second plurality of arms 144.

The radially outward force F_{R} exerted against the first arm of the second plurality of arms 144 of the second iris 140 may urge and/or bias the second circumferential ring 142 in the first circumferential direction (e.g., clockwise) when the first inner hinge point of the first arm of the second plurality of arms 144 of the second iris 140 is disposed on the second side of the first radius 104. When the first inner hinge point of the first arm of the second plurality of arms 144 of the second iris 140 is disposed on the first side of the first radius, the first angle is positive (e.g., opens to the right or clockwise from the first radius). As such, when the first inner hinge point of the first arm of the second plurality of arms 144 of the second iris 140 is disposed on the second side of the first radius and/or when the first angle is positive, the radially outward force F_{R} exerted against the first arm of the second plurality of arms 144 of the second iris 140 may urge and/or bias the second iris 140 and/or the second plurality of arms 144 towards the second configuration and/or a locked configuration. In some embodiments, the radially outward force F_{R} exerted against the second plurality of arms 144 of the second iris 140 may urge and/or bias the second circumferential ring 142 in a second direction (e.g., the first circumferential direction, clockwise) in the second configuration and/or the locked configuration of the second plurality of arms 144.

Clockwise rotation of the second lever 141 and/or the second circumferential ring 142 causes a radially outermost end and/or the first living hinge of each arm of the second plurality of arms 144 to shift in the clockwise direction as well. Since the pivot end of each arm of the second plurality of arms 144 is held in a fixed position relative to the housing 110 by engagement of the aperture with the plurality of projections 160 and/or the second plurality of projections, the medial portion of each arm of the second plurality of arms 144 will pivot about the plurality of projections 160 and/or the second plurality of projections as the second lever 141 and/or the second circumferential ring 142 is rotated. The first living hinge and the second living hinge of each arm of the second plurality of arms 144 may resiliently flex, deflect, and/or bend at the first outer hinge point and the first inner hinge point, respectively, to facilitate a pivoting movement of the second plurality of arms 144 around the pivot point and/or the aperture and/or the plurality of projections 160 and/or the second plurality of projections. As the second lever 141 and/or the second circumferential ring 142 is rotated clockwise, each arm of the second plurality of arms 144 may be prevented from rotation commensurately and/or equally around the central longitudinal axis 102 due to the arm being fixed in position by the plurality of projections 160 and/or the second plurality of projections. Lengths of the various portions of each arm of the second plurality of arms 144 remain fixed and/or constant, wherein the first living hinge and the second living hinge cooperate with the elongated link portion of each arm connecting the medial portion to the second circumferential ring 142 to facilitate movement and/or translation of the second plurality of arms 144 from the first configuration toward and/or to the second configuration.

In some embodiments, each arm of the second plurality of arms 144 of the second iris 140 may engage at least one other arm of the second plurality of arms 144 of the second iris 140 as the first plurality of arms 144 shifts from the first configuration of the second plurality of arms 144 to the second configuration of the second plurality of arms 144. As the second lever 141 and/or the second circumferential ring 142 is rotated clockwise and/or as the second plurality of arms 144 shifts from the first configuration to the second configuration, the medial portion of each arm of the second plurality of arms 144 may shift radially inward relative to the second circumferential ring 142 and/or toward the central longitudinal axis 102, thereby changing the size of the second central opening 146 defined by the second plurality of arms 144. In the first configuration, the elongated link portion of each arm of the second plurality of arms 144 connected to the second circumferential ring 142 by the first living hinge and/or extending between the first living hinge and the second living hinge may be oriented at an oblique angle to the second circumferential ring 142. As the second lever 141 and/or the second circumferential ring 142 is rotated clockwise toward the second configuration, the elongated link portion of each arm of the second plurality of arms 144 connected to the second circumferential ring 142 by the first living hinge and/or extending between the first living hinge and the second living hinge may shift toward an angle that is closer to normal or perpendicular to the second circumferential ring 142 (e.g., wherein the first axis is aligned with the first radius).

By using living hinges to shift the irises and/or the pluralities of arms from the first configuration toward and/or to the second configuration (and vice versa), the complexity and high part count of traditional stent compression devices may be reduced. As such, the cost of such devices may also be reduced. Additionally, in some embodiments, the device 100 may permit a stent to be loaded into a sheath without moving or advancing the stent through the device 100 multiple times, thereby reducing the number of steps required to sheath the stent, reducing or eliminating multiple compression steps, and/or reducing opportunity for damage to the stent. In some embodiments, the device 100 may be reusable following suitable sterilization techniques. In some embodiments, the device 100 may be disposable and/or may be classified or used as a single-use device.

Additionally, it is contemplated that the device 100 may include additional irises, intervening spacer plates, etc. to accommodate a stent of longer length and/or varying outer diameter (in a first configuration and/or in a compressed configuration). For example, in some embodiments, the second size of the first central opening 126 may be the same as second size of the second central opening 146, and additional central openings of additional irises may have a second size that is the same as the second size of the first central opening 126 and/or the second central opening 146. In some embodiments, the second size of the first central opening 126 may be different from the second size of the second central opening 146, and additional central openings of additional irises may have a second size that is the same as the second size of the first central opening 126 and the second central opening 146, the additional central openings of additional irises may have a second size that is the same as the second size of one of the first central opening 126 and the second central opening 146, or the additional central openings of additional irises may have a second size that is the different from the second size of the first central opening 126 and the second central opening 146. Other configurations are also contemplated.

In some embodiments, the first iris 120 and/or the medial portions of the first plurality of arms 124, the second iris 140 and/or the medial portions of the second plurality of arms 144, and/or additional irises and/or medial portions of the pluralities of arms thereof could be shaped or tapered axially to accommodate stent having outer surfaces that are curved or tapered in an axial direction.

In some embodiments, the first iris 120 may have a first axial thickness and the second iris 140 may have a second axial thickness. In some embodiments, the first axial thickness may be equal to the second axial thickness. In some embodiments, the first axial thickness may be different from the second axial thickness. In some embodiments, the first axial thickness may be less than the second axial thickness. In some embodiments, the first axial thickness may be greater than the second axial thickness. In some embodiments, additional irises may each have an axial thickness. The axial thickness of any additional iris may be equal to, more than, or less than the first axial thickness and/or the second axial thickness, as desired, to accommodate stents having different sizes, different lengths, and/or different radial compression requirements along their length.

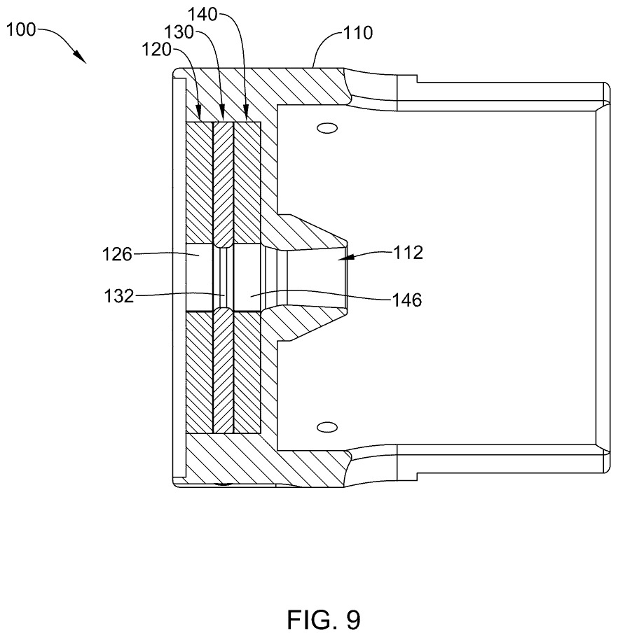

FIGS. 9-13 are partial schematic cross-sectional views of the device 100 showing selected aspects of a method of radially compressing a stent 200 and/or a method of loading a stent 200 into a sheath 300. The device 100 may generally include elements and/or features as described herein. For improved clarity and understanding, some elements or features of the device 100 are not shown, are not shown in their entirety, or are shown schematically.

FIG. 9 illustrates a partial schematic cross-sectional view of the device 100 for radially compressing a stent 200 as described herein. In at least some embodiments, the device 100 may be used to load the stent 200 into a sheath 300. The device 100 may include the housing 110 defining the central opening 112. In some embodiments, at least a portion of the central opening 112 may have an inner diameter that is tapered radially outward in an axial direction. In some embodiments, the central opening 112 may have an inner diameter that is generally constant. In some embodiments, the central opening 112 may have an inner diameter that is generally constant along a portion of its axial length and tapered along a different portion of its axial length. Other configurations are also contemplated.

The device 100 may comprise the first iris 120 including the first circumferential ring 122 and the first plurality of arms 124 (e.g., FIG. 3) extending radially inward from the first circumferential ring 122 to define the first central opening 126. The first central opening 126 may have a first size in a first configuration of the first plurality of arms 124 and a second size in a second configuration of the first plurality of arms 124. The device 100 may include the spacer plate 130 including and/or defining the central opening 132 therein. In at least some embodiments, the central opening 132 may have a generally fixed size. The device 100 may include the second iris 140 including the second plurality of arms 144 (e.g., FIG. 3) defining the second central opening 146. The second central opening 146 may have a first size in a first configuration of the second plurality of arms 144 and a second size in a second configuration of the second plurality of arms 144.

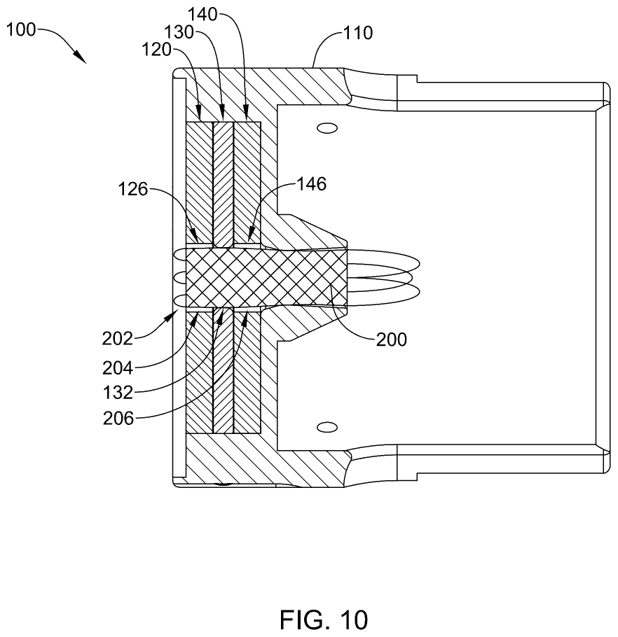

A method of radially compressing the stent 200 and/or a method of loading the stent 200 into the sheath 300 may comprise inserting a stent 200 in a first configuration into the first iris 120 such that a first end portion 202 of the stent 200 is disposed outside of the first iris 120, as seen in FIG. 10. In some embodiments, the method may comprise inserting the stent 200 in the first configuration into the first central opening 126 of the first iris 120 such that a first portion 204 of the stent 200 is disposed within the first iris 120. In at least some embodiments, the first portion 204 of the stent 200 is disposed immediately adjacent the first end portion 202 of the stent 200. In some embodiments, in the first configuration, the stent 200 may be in an expanded configuration. In some embodiments, in the first configuration, the stent 200 may be in a partially collapsed state. For example, in some embodiments, the device 100 may include a loading funnel (not shown) adapted to partially collapse the stent 200 as the stent 200 is inserted into the first central opening 126 of the first iris 120 and/or the central opening 112 of the housing 110. In some embodiments, the loading funnel may be configured to releasably attach to the housing 110. For example, in the view shown in FIG. 10, the loading funnel may be configured to be inserted into the housing 110 from the right side of the view until at least a portion of the loading funnel is positioned adjacent to and/or is engaged with the housing 110 proximate the central opening 112. Other configurations are also contemplated. In some embodiments, the method may include inserting the stent 200 into and/or through the central opening 112 of the housing 110 before inserting the stent 200 in the first configuration into first central opening 126 of the first iris 120.

In some embodiments, the method may comprise inserting the stent 200 in the first configuration into the first iris 120 and a second iris 140 axially offset from the first iris 120 and/or a second central opening 146 of the second iris 140 axially offset from the first iris 120 such that a second portion 206 of the stent 200 is disposed within the second iris 140. As discussed herein, the second iris 140 may include a second circumferential ring 142 and a second plurality of arms 144 extending radially inward from the second circumferential ring 142 to define the second central opening 146. In some embodiments including the second iris 140, the method may include inserting the stent 200 in the first configuration into and/or through the central opening 132 of the spacer plate 130. Additionally, while not expressly illustrated, the method may include inserting the stent 200 into and/or through the cover plate opening 156 of the cover plate 150 (e.g., FIG. 3).

The stent 200 may include an expandable framework defining a central lumen which, in some embodiments, may be substantially cylindrical. In some embodiments, the expandable framework may have a substantially circular cross-section. In some embodiments, the expandable framework can have a non-circular (e.g., D-shaped, elliptical, etc.) cross-section. Some suitable but non-limiting examples of materials that may be used to form the expandable framework, including but not limited to metals and metal alloys, composites, ceramics, polymers, and the like, are described below.

The stent 200 and/or the expandable framework may be configured to shift from a collapsed configuration to an expanded configuration. In some embodiments, the expandable framework may be self-expanding. In some embodiments, the expandable framework may be self-biased toward the expanded configuration. In some embodiments, the expandable framework may be mechanically expandable. In some embodiments, the expandable framework may be balloon expandable. Other configurations are also contemplated.

In some embodiments, the stent 200 may be a part of a replacement heart valve implant. It will be appreciated that the replacement heart valve implant can be any type of heart valve (e.g., a mitral valve, an aortic valve, etc.). The replacement heart valve implant can be configured to allow one-way flow through the replacement heart valve implant from an inflow end to an outflow end. In some embodiments of a replacement heart valve implant, the stent 200 and/or the expandable framework may define a lower crown proximate an inflow end of the replacement heart valve implant, an upper crown proximate an outflow end of the replacement heart valve implant, and a plurality of stabilization arches extending downstream from the outflow end.

In some embodiments, the replacement heart valve implant may include a plurality of valve leaflets disposed within the central lumen. The plurality of valve leaflets may be coupled, secured, and/or fixedly attached to the stent 200 and/or the expandable framework. In some embodiments, the plurality of valve leaflets can be integrally formed with each other, such that the plurality of valve leaflets is formed as a single unitary and/or monolithic unit. In some embodiments, the plurality of valve leaflets may be formed integrally with other structures such as an inner skirt and/or an outer skirt, base structures, liners, or the like. The plurality of valve leaflets may be configured to substantially restrict fluid from flowing through the replacement heart valve implant in a closed position. For example, in some embodiments, free edges of the plurality of valve leaflets may move into coaptation with one another in the closed position to substantially restrict fluid from flowing through the replacement heart valve implant. The free edges of the plurality of valve leaflets may be move apart from each other in an open position to permit fluid flow through the replacement heart valve implant.

In some embodiments, the replacement heart valve implant may include an inner skirt. The inner skirt may be disposed on and/or extend along an inner surface of the stent 200 and/or the expandable framework. In at least some embodiments, the inner skirt may be fixedly attached to the stent 200 and/or the expandable framework. The inner skirt may direct fluid, such as blood, flowing through the replacement heart valve implant toward the plurality of valve leaflets. In at least some embodiments, the inner skirt may be fixedly attached to and/or integrally formed with the plurality of valve leaflets. The inner skirt may ensure the fluid flows through the central lumen and does not flow around the plurality of valve leaflets when they are in the closed position.

In some embodiments, the replacement heart valve implant may include an outer skirt. In some embodiments, the outer skirt may be disposed on and/or extend along an outer surface of the stent 200 and/or the expandable framework. In some embodiments, the outer skirt may be disposed between the stent 200 and/or the expandable framework and native tissue in order to prevent fluid, such as blood, flowing around the stent 200 and/or the expandable framework in a downstream direction so as to ensure that the plurality of valve leaflets can stop the flow of fluid when in the closed position.

In some embodiments, the plurality of valve leaflets may be comprised of a polymer, such as a thermoplastic polymer. In some embodiments, the plurality of valve leaflets may include at least 50 percent by weight of a polymer. In some embodiments, the plurality of valve leaflets may be formed from bovine pericardial or other living tissue. Other configurations and/or materials are also contemplated.

In some embodiments, the inner skirt and/or the outer skirt may include a polymer, such as a thermoplastic polymer. In some embodiments, the inner skirt and/or the outer skirt may include at least 50 percent by weight of a polymer. In some embodiments one or more of the plurality of valve leaflets, the inner skirt, and/or the outer skirt may be formed of the same polymer or polymers. In some embodiments, the polymer may be a polyurethane. In some embodiments, the inner skirt and/or the outer skirt may be substantially impervious to fluid. In some embodiments, the inner skirt and/or the outer skirt may be formed from a thin tissue (e.g., bovine pericardial, etc.). In some embodiments, the inner skirt and/or the outer skirt may be formed from a coated fabric material. In some embodiments, the inner skirt and/or the outer skirt may be formed from a nonporous and/or impermeable fabric material. Other configurations are also contemplated. Some suitable but non-limiting examples of materials that may be used to form the inner skirt and/or the outer skirt including but not limited to polymers, composites, and the like, are described below.

In some embodiments, the stent 200 and/or the replacement heart valve implant may have an outer extent of about 23 millimeters (mm), about 25 mm, about 27 mm, about 30 mm, etc. in an unconstrained configuration (e.g., in the expanded configuration). In some embodiments, the stent 200 and/or the replacement heart valve implant may have an outer extent of about 10 mm, about 9 mm about 8 mm, about 7 mm, about 6 mm, etc. in the collapsed configuration. Other configurations are also contemplated.

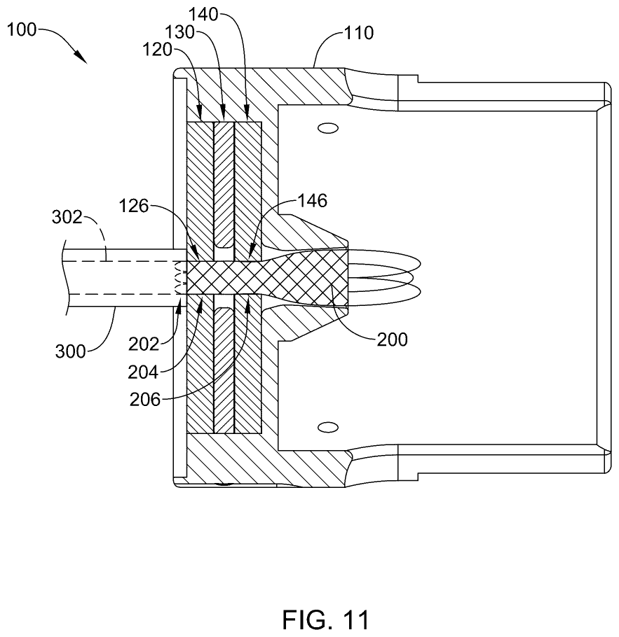

In some embodiments, the method may comprise rotating the first circumferential ring 122 of the first iris 120 relative to the housing 110 disposed about the first circumferential ring 122 to shift the first plurality of arms 124 from the first configuration to the second configuration, as seen in FIG. 11, wherein the first central opening 126 has a first size in the first configuration (e.g., FIG. 10) and a second size in the second configuration less than the first size (e.g., FIG. 11). In some embodiments, the method may comprise rotating the first circumferential ring 122 of the first iris 120 clockwise relative to the housing 110 disposed about the first circumferential ring 122 to shift the first plurality of arms 124 from the first configuration to the second configuration. In the second configuration of the first plurality of arms 124, the first portion 204 of the stent 200 disposed within the first iris 120 and/or the first central opening 126 may be radially compressed and exert a radially outward force F_{R} (e.g., FIG. 8A) against the first plurality of arms 124. In at least some embodiments, in the second configuration of the first plurality of arms 124, the radially outward force F_{R} (e.g., FIG. 8A) against the first plurality of arms 124 may urge and/or bias the first plurality of arms 124 toward the second configuration of the first plurality of arms 124.

In some embodiments, the method may comprise rotating the second circumferential ring 142 of the second iris 140 relative to the housing 110 to shift the second plurality of arms 144 from a first configuration to a second configuration, wherein the second central opening 146 has a first size in the first configuration and a second size in the second configuration less than the first size. In some embodiments, the method may comprise rotating the second circumferential ring 142 of the second iris 140 clockwise relative to the housing 110 to shift the second plurality of arms 144 from a first configuration to a second configuration. In the second configuration of the second plurality of arms 144, the second portion 206 of the stent 200 disposed within the second iris 140 and/or the second central opening 146 may be radially compressed and exert a radially outward force against the second plurality of arms 144. In at least some embodiments, in the second configuration of the second plurality of arms 144, the radially outward force against the second plurality of arms 144 may urge and/or bias the second plurality of arms 144 toward the second configuration of the second plurality of arms 144.

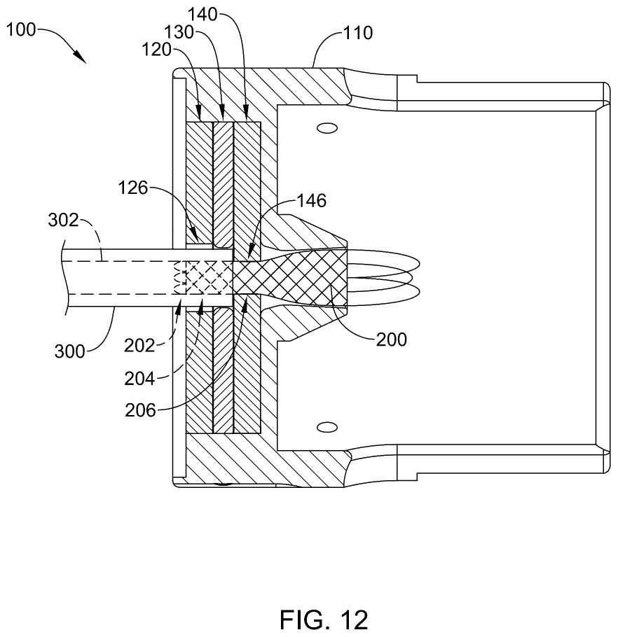

In some embodiments, the method may comprise positioning a sheath 300 proximate the first iris 120 with the first plurality of arms 124 in the second configuration and the first portion of the stent 200 radially compressed within the first iris 120 such that the first end portion 202 of the stent 200 is disposed within a lumen 302 of the sheath 300, as seen in FIG. 11. After positioning the sheath 300 proximate the first iris 120, the method may comprise rotating the first circumferential ring 122 of the first iris 120 relative to the housing 110 to shift the first plurality of arms 124 from the second configuration to the first configuration. In some embodiments, the method may comprise rotating the first circumferential ring 122 of the first iris 120 counterclockwise relative to the housing 110 to shift the first plurality of arms 124 from the second configuration to the first configuration. The method may further comprise moving the sheath 300 into the first iris 120 over the stent 200 such that the first portion 204 of the stent 200 that was disposed within the first iris 120 is disposed within the lumen 302 of the sheath 300, as seen in FIG. 12. In some embodiments, the sheath 300 has an inner diameter less than an outer diameter of the first portion 204 of the stent 200 when the stent 200 is in the first configuration. As such, radial compression of the stent 200 is required in order to move the stent 200 into the lumen 302 of the sheath 300.

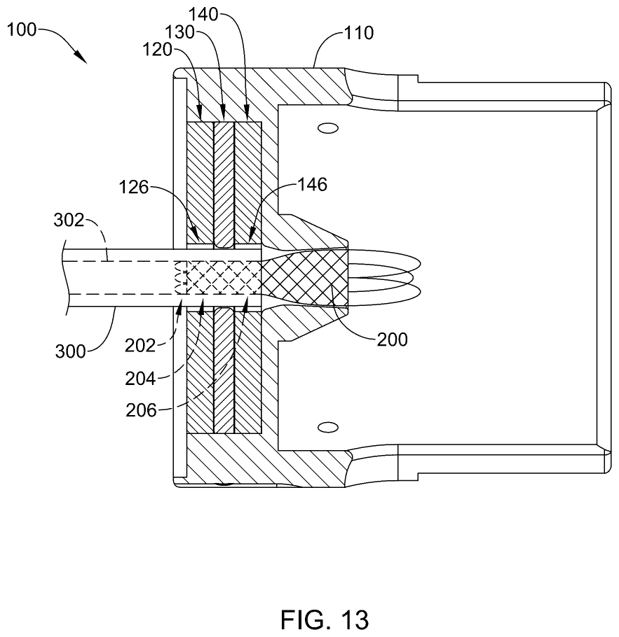

In some embodiments, after moving the sheath 300 into the first iris 120 over the stent 200 such that the first portion 204 of the stent 200 that was disposed within the first iris 120 is disposed within the lumen 302 of the sheath 300, the method may further comprise rotating the second circumferential ring 142 of the second iris 140 relative to the housing 110 to shift the second plurality of arms 144 of the second iris 140 from the second configuration to the first configuration. In some embodiments, the method may comprise rotating the second circumferential ring 142 of the second iris 140 counterclockwise relative to the housing 110 to shift the second plurality of arms 144 of the second iris 140 from the second configuration to the first configuration. The method may further comprise moving the sheath 300 into the second iris 140 over the stent 200 in the compressed configuration such that the second portion 206 of the stent 200 that was disposed within the second iris 140 is disposed within the lumen 302 of the sheath 300, as seen in FIG. 13.

In some embodiments, wherein the device 100 includes additional irises, the process described above with respect to moving the sheath 300 into the first iris 120 and the second iris 140 may be repeated as necessary to move the sheath 300 over additional portions of the stent 200 disposed within those irises.

In some embodiments, if additional length of the stent 200 needs to be moved into the sheath 300, the stent 200 may be advanced through the central opening 112 of the housing 110 and the first iris 120 and the second iris 140 may be again shifted from the first configuration to the second configuration and the process may be repeated.

In some embodiments, after moving the sheath 300 into the second iris 140 over the stent 200 such that the second portion 206 of the stent 200 that was disposed within the second iris 140 is disposed within the lumen of the sheath 300, the sheath 300 and the first and second portions of the stent 200 that were disposed within the first and second irises, respectively, may be advanced through the central opening 112 of the housing 110, and a separate sheath may be translated over an uncovered portion of the stent 200 toward the sheath 300 to cover at least some of the uncovered portion of the stent 200 that remains outside of the sheath 300. Other configurations are also contemplated.

The materials that can be used for the various components of the device and the various elements thereof disclosed herein may include those commonly associated with medical devices and devices used and/or associated with medical devices. For simplicity purposes, the following discussion refers to the system. However, this is not intended to limit the devices and methods described herein, as the discussion may be applied to other elements, members, components, or devices disclosed herein, such as, but not limited to, the stent, the expandable framework, the first and/or second plurality of arms, the circumferential ring(s), the housing, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A device for radially compressing a stent, comprising:
a housing including a central opening; and
a first iris positioned adjacent the housing;
wherein the first iris includes a first circumferential ring positioned coaxially relative to the central opening and a first plurality of arms extending radially inward from the first circumferential ring;
wherein each arm of the first plurality of arms defines a first outer hinge point and a first inner hinge point disposed radially inward relative to the first outer hinge point;
wherein rotation of the first circumferential ring in a first circumferential direction is configured to shift the first inner hinge point of a first arm of the first plurality of arms from a first side of a first radius extending through a central longitudinal axis of the first iris and the first outer hinge point of the first arm of the first plurality of arms to a second side of the first radius.

2. The device of claim 1, wherein a radially outward force exerted against the first arm of the first plurality of arms urges the first circumferential ring in a second circumferential direction opposite the first circumferential direction when the first inner hinge point of the first arm of the first plurality of arms is disposed on the first side of the first radius.

3. The device of claim 1, wherein a radially outward force exerted against the first arm of the first plurality of arms urges the first circumferential ring in the first circumferential direction when the first inner hinge point of the first arm of the first plurality of arms is disposed on the second side of the first radius.

4. The device of claim 1, wherein the first inner hinge point of the first arm of the first plurality of arms is configured to move in an opposite circumferential direction from the first circumferential ring.

5. The device of claim 1, wherein a first axis extending through the first inner hinge point of the first arm of the first plurality of arms and the first outer hinge point of the first arm of the first plurality of arms defines a first angle from the first radius to the first axis;
wherein when the first inner hinge point of the first arm of the first plurality of arms is disposed on the first side of the first radius the first angle is negative, and when the first inner hinge point of the first arm of the first plurality of arms is disposed on the second side of the first radius the first angle is positive.

6. The device of claim 1, wherein the first plurality of arms defines a first central opening positioned coaxially relative to the central opening of the housing;
wherein rotation of the first circumferential ring relative to the housing changes a size of the first central opening.

7. The device of claim 1, wherein the first outer hinge point of each arm of the first plurality of arms is defined by a first living hinge coupling the arm to the first circumferential ring.

8. The device of claim 1, wherein the first inner hinge point is defined by a second living hinge disposed at an opposite end of an elongated link portion extending from the first living hinge to the second living hinge.

9. The device of claim 1, wherein the first plurality of arms is monolithically formed with the first circumferential ring from a single piece of material.

10. A device for radially compressing a stent, comprising:
a housing including a central opening; and
a first iris positioned adjacent the housing;
wherein the first iris includes a first circumferential ring positioned coaxially relative to the central opening of the housing and a first plurality of arms extending radially inward from the first circumferential ring;
wherein the first plurality of arms defines a first central opening positioned coaxially relative to the central opening of the housing;
wherein the first plurality of arms is configured to shift between a first configuration of the first plurality of arms and a second configuration of the first plurality of arms via rotation of the first circumferential ring relative to the housing;
wherein a radially outward force exerted against the first plurality of arms biases the first circumferential ring in a first direction in the first configuration of the first plurality of arms and biases the first circumferential ring in a second direction opposite the first direction in the second configuration of the first plurality of arms.

11. The device of claim 10, wherein in the first configuration of the first plurality of arms, the first plurality of arms defines a first size of the first central opening, and in the second configuration of the first plurality of arms, the first plurality of arms defines a second size of the first central opening less than the first size of the first central opening.

12. The device of claim 10, wherein each arm of the first plurality of arms engages at least one other arm of the first plurality of arms as the first plurality of arms shifts from the first configuration of the first plurality of arms to the second configuration of the first plurality of arms.

13. The device of claim 10, further comprising a second iris axially offset from the first iris;
wherein the second iris includes a second circumferential ring positioned coaxially relative to the central opening of the housing and a second plurality of arms extending radially inward from the second circumferential ring;
wherein the second plurality of arms defines a second central opening positioned coaxially relative to the central opening of the housing;
wherein the second plurality of arms is configured to shift between a first configuration of the second plurality of arms and a second configuration of the second plurality of arms via rotation of the second circumferential ring relative to the housing;
wherein a radially outward force exerted against the second plurality of arms biases the second circumferential ring in the first direction in the first configuration of the second plurality of arms and biases the second circumferential ring in the second direction in the second configuration of the second plurality of arms.

14. The device of claim 13, wherein in the first configuration of the second plurality of arms, the second plurality of arms defines a first size of the second central opening, and in the second configuration of the second plurality of arms, the second plurality of arms defines a second size of the second central opening less than the first size of the second central opening.

15. A method of loading a stent into a sheath, comprising:
inserting a stent in a first configuration into a first iris such that a first end portion of the stent is disposed outside of the first iris, wherein the first iris includes a first circumferential ring and a first plurality of arms extending radially inward from the first circumferential ring to define a first central opening; and
rotating the first circumferential ring relative to a housing disposed about the first circumferential ring to shift the first plurality of arms from a first configuration to a second configuration, wherein the first central opening has a first size in the first configuration and a second size in the second configuration less than the first size;
wherein in the second configuration of the first plurality of arms, a first portion of the stent disposed within the first iris is radially compressed and exerts a radially outward force against the first plurality of arms, wherein the first portion of the stent is disposed immediately adjacent the first end portion of the stent;
wherein in the second configuration of the first plurality of arms, the radially outward force against the first plurality of arms biases the first plurality of arms toward the second configuration of the first plurality of arms.

16. The method of claim 15, further comprising:
positioning a sheath proximate the first iris with the first plurality of arms in the second configuration and the first portion of the stent radially compressed within the first iris such that the first end portion of the stent is disposed within the sheath;
rotating the first circumferential ring relative to the housing to shift the first plurality of arms from the second configuration of the first plurality of arms to the first configuration of the first plurality of arms; and
moving the sheath into the first iris over the stent such that the first portion of the stent that was disposed within the first iris is disposed within the sheath.

17. The method of claim 16, wherein the sheath has an inner diameter less than an outer diameter of the first portion of the stent when the stent is in the first configuration.

18. The method of claim 15, wherein inserting the stent further includes inserting the stent in the first configuration into the first iris and a second iris axially offset from the first iris, wherein the second iris includes a second circumferential ring and a second plurality of arms extending radially inward from the second circumferential ring to define a second central opening.

19. The method of claim 18, further comprising:
rotating the second circumferential ring relative to the housing to shift the second plurality of arms from a first configuration of the second plurality of arms to a second configuration of the second plurality of arms, wherein the second central opening has a first size in the first configuration and a second size in the second configuration less than the first size;
wherein in the second configuration of the second plurality of arms, a second portion of the stent disposed within the second iris is radially compressed and exerts a radially outward force against the second plurality of arms;
wherein in the second configuration of the second plurality of arms, the radially outward force against the second plurality of arms biases the second plurality of arms toward the second configuration of the second plurality of arms.

20. The method of claim 19, further comprising:
positioning a sheath proximate the first iris with the first plurality of arms in the second configuration of the first plurality of arms and the first portion of the stent radially compressed within the first iris and the second portion of the stent radially compressed within the second iris such that the first end portion of the stent is disposed within the sheath;
rotating the first circumferential ring relative to the housing to shift the first plurality of arms from the second configuration of the first plurality of arms to the first configuration of the first plurality of arms;
moving the sheath into the first iris over the stent such that the first portion of the stent that was disposed within the first iris is disposed within the sheath;
rotating the second circumferential ring relative to the housing to shift the second plurality of arms from the second configuration of the second plurality of arms to the first configuration of the second plurality of arms; and
moving the sheath into the second iris over the stent such that the second portion of the stent that was disposed within the second iris is disposed within the sheath.
